(19) United States
(12) Patent Application Publication
(10) Pub. No.: US 2024/0238008 A1
(43) Pub. Date: Jul. 18, 2024

(54) CANNULA ASSEMBLY

(71) Applicant: B. Braun Melsungen AG, Melsungen (DE)

(72) Inventors: Lars Grosser, Melsungen (DE); André Weiss, Guxhagen (DE); Thomas Riemann, Grossalmerode (DE)

(21) Appl. No.: 18/560,160

(22) PCT Filed: May 10, 2022
(86) PCT No.: PCT/EP2022/062538

§ 371 Date: Nov. 10, 2023

(30) Foreign Application Priority Data
May 11, 2021 (DE) 10 2021 204 780.7

(51) Int. Cl. A61B 17/34; A61B 17/00; A61M 25/09

(52) U.S. Cl. CPC A61B 17/3423; A61M 25/09; A61B 2017/00862; A61B 2017/347

(57) ABSTRACT

A cannula assembly, which can be used in a catheterization process, has an elongate cannula tube with a proximal inlet opening, a distal outlet opening, a lumen extending continuously between the inlet opening and outlet opening, and a sharpened cannula tip delimiting the outlet opening. The cannula tube is configured to receive and guide a flexible medical functional section, which is insertable into the lumen and movable along the lumen. A blocking device with a blocking element is assigned to the lumen. The blocking element is configured to interact with the flexible medical functional section and, depending on a movement direction of the flexible medical functional section, is automatically transferred between a blocking position, in which the blocking element blocks a withdrawal movement of the flexible medical functional section by acting on the functional section, and a release position, in which the blocking element releases an advancing movement.

### CROSS-REFERENCE TO RELATED APPLICATIONS

This application is the United States national stage entry of International Application No. PCT/EP2022/062538, filed on May 10, 2022, which claims priority to German Application No. 10 2021 204 780.7, filed on May 11, 2021. The contents of International Application No. PCT/EP2022/062538 and German Application No. 10 2021 204 780.7 are incorporated by reference herein in their entireties.

### FIELD

The invention relates to a cannula assembly, having an elongate cannula tube with a proximal inlet opening, a distal outlet opening, a lumen extending continuously between the inlet opening and the outlet opening, and with a sharpened cannula tip delimiting the outlet opening, wherein the cannula tube is configured to receive and guide a flexible medical functional section, which can be inserted into the lumen and is movable along the lumen.

### BACKGROUND

Cannula assemblies are generally known in medical practice and are used, inter alia, for puncturing human or animal body tissue and for subsequently introducing and/or removing fluids.

For this purpose, conventional cannula assemblies have an elongate cannula tube with a proximal inlet opening and a distal outlet opening. A continuously extending lumen is provided between the inlet opening and the outlet opening. The outlet opening is delimited by a sharpened cannula tip, which is frequently also referred to as a sharpening or cannula eyelet. The sharpened cannula tip makes it easier to puncture the body tissue.

Furthermore, different medical applications are known in which the cannula tube serves as a mandril for a medical functional section to be inserted into the body. The medical functional section can be, for example, an elongate probe, a light guide, a guide wire or the like.

One important application is the insertion of a central venous catheter by what is referred to as the Seldinger technique. The Seldinger technique comprises inserting a guide wire into the inlet opening of the cannula tube, advancing the guide wire in the distal advancing direction through the lumen and exiting the guide wire from the outlet opening.

### SUMMARY

It is an object of the present invention to provide a cannula assembly of the type mentioned at the beginning which provides increased patient safety.

This object is achieved in that a blocking device with at least one blocking element is provided and assigned to the lumen, wherein the at least one blocking element is configured to interact with the functional section and, depending on a movement direction of the functional section, is automatically transferred between a blocking position, in which the at least one blocking element blocks a proximally directed withdrawal movement of the functional section by acting on the functional section, and a release position, in which the at least one blocking element releases a distally directed advancing movement. The invention is based on the finding that the medical functional section may be damaged or even severed during a proximal withdrawal movement, i.e. as it is being pulled out through the cannula tube. This is because, during the pulling out process, the functional section may under some circumstances contact the sharpened cannula tip delimiting the outlet opening, the sharpened cannula tip being able to act as a type of blade on the functional section. This may lead to serious complications. Under some circumstances, an emergency operation may be necessary to remove the partially severed functional section from the body. To counteract all of this, the solution according to the invention provides the blocking device. The blocking device blocks the movability of the functional section in the proximal direction, and therefore, although the functional section can be readily advanced in the distal direction, it cannot be pulled out, or can be only to a limited extent, in the proximal direction. This counteracts damage to the functional section by the sharpened cannula tip and ultimately achieves improved patient safety. The blocking device preferably interacts mechanically with the functional section, and therefore it is also possible to speak of a mechanical blocking device. Alternatively or additionally, the blocking device can interact magnetically and/or electrically with the functional section. For blocking the movability of the functional section, the blocking device has the at least one blocking element assigned to the lumen. The at least one blocking element can be automatically transferred between the release position and the blocking position under the action of the functional section inserted into the lumen and depending on the respective movement direction. The transfer between the release position and the blocking position can comprise an elastic deformation, in particular bending deformation, and/or a, in particular translational and/or rotational, rigid body movement of the at least one blocking element. In the blocking position, the at least one blocking element acts on the functional section, thus preventing a proximal movement or at any rate a further proximal movement of the functional section through the lumen. Preferably, the at least one blocking element acts in the blocking position on an outer surface of the functional section with a force fit and/or form fit. Automatically means that the transfer or the change between the release position and the blocking position, depending on the movement direction of the functional section, takes place without engagement from the outside. Preferably, in the release position, the at least one blocking element is moved radially outward relative to the lumen. Furthermore preferably, in the blocking position, the at least one blocking element is moved radially inward relative to the lumen. In a use state of the cannula assembly, in which the medical functional section is inserted into the lumen, the at least one blocking element preferably interacts mechanically with the outer surface of the functional section. Preferably, the at least one blocking element acts in the blocking position proximally along a longitudinal axis of the lumen—and therefore along a longitudinal axis of the functional section—on the functional section with a force fit and/or form fit, more precisely: on the outer surface and/or circumferential surface thereof. In the release position, this action is canceled. The medical functional section is not part of the cannula assembly according to the invention. Embodiments of the cannula assembly can comprise a/the medical functional section. The medical functional section is elongate and flexible and can be in particular a medical wire, for example a guide wire for the catheter insertion, a medical probe, a light guide, a thread or wire arrangement which can in each case be twined and/or twisted, or the like.

In one embodiment, the blocking device is integrated in the cannula tube, or the blocking device is integrated in a cannula attachment to which a cannula end of the cannula tube that faces away from the cannula tip is attached, or the blocking device has a device housing in which the at least one blocking element is accommodated and which is attached and/or attachable to the cannula end and/or to the cannula attachment. Integration of the blocking device in the cannula tube permits a particularly compact construction of the cannula assembly. In this case, the at least one blocking element can be formed by a tube wall portion of a tube wall of the cannula tube. Alternatively, the at least one blocking element can be manufactured separately from the cannula tube and subsequently inserted and/or integrated therein. Integration of the blocking device in the cannula attachment provides comparatively more construction space and to this extent provides further structural degrees of freedom with regard to the spatial and physical configuration of the blocking device and/or of the at least one blocking element. The cannula attachment may also be referred to as a cannula hub and is fastened proximally to the cannula tube. Apart from the integrated blocking device, the cannula attachment preferably has a conventional construction, so that there can be in particular a proximally arranged connection port for connecting a fluid-guiding component to the cannula attachment. The connection port can be configured, for example, as a Luer connector. The cannula attachment is preferably joined non-releasably together with the cannula tube. In this embodiment of the invention, the cannula attachment is part of the cannula assembly. Said device housing permits a particularly flexible use of the blocking device. The device housing is attached and/or attachable proximally to the cannula tube. If the cannula assembly has a cannula attachment, the device housing is attached and/or attachable proximally to the cannula attachment. The respective attachment is preferably releasable. If there is a cannula attachment and/or a device housing, the functional section—in a use state of the cannula assembly—extends longitudinally through a respective lumen of the catheter attachment and/or of the device housing.

In one embodiment, the at least one blocking element is a blocking pawl which is spring-elastically movable and/or pivotally movable between the release position and the blocking position and has a blocking edge which is oriented in the distal direction and, during an advancing movement, interacts relatively slidably with an outer surface of the functional section and, during a withdrawal movement, is caused by frictional force to be positioned against the outer surface and blocks the withdrawal movement with a force fit and/or form fit. This embodiment of the invention permits a particularly simple construction of the blocking device. The blocking pawl can be integrated in the cannula tube. Alternatively, the blocking pawl can be integrated in an optionally present cannula attachment of the cannula assembly. If the blocking device has a device housing, the blocking pawl is preferably accommodated therein. The blocking pawl interacts mechanically by means of its blocking edge with the outer surface of the functional section.

The outer surface may also be referred to as the circumferential surface. To permit a direction-dependent movement between the release position and the blocking position, the blocking edge is oriented in the distal direction. Owing to said distal orientation, a frictional force occurring between the blocking edge and the outer surface because of the movement causes either a force component acting outward or inward in the radial direction of the lumen. The force component acting radially outward occurs during the distal advancing movement. By this means, the blocking edge is pressed radially outward under the action of the outer surface. The force component acting radially inward occurs during the proximal withdrawal movement. By this means, the blocking edge is pressed radially inward onto the functional section, more precisely: onto the circumferential surface thereof. To this extent, self-reinforcing and/or self-locking may also be spoken of. The force component acting radially inward results in a force fit and/or form fit between the blocking edge and the outer surface.

In one embodiment, the blocking pawl is configured in the manner of a bending spring, wherein the bending spring is secured at one end relative to the lumen and at the other end has the blocking edge. The bending spring is spring-elastically movable between the release position and the blocking position. In particular, a pivoting or otherwise movable mounting of the blocking pawl can thereby be dispensed with. This permits a further simplification of the design.

In one embodiment, the blocking pawl is formed by a tube wall portion, that is released in the form of a tongue, of a tube wall of the cannula tube. This permits a particularly compact and simple construction of the cannula assembly. Firstly, in this embodiment of the invention, a special construction space for the at least one blocking element, more precisely: the blocking pawl, is not required. Secondly, additional components for forming the, preferably mechanical, blocking device are not required. On the contrary, the cannula tube itself forms the, preferably mechanical, blocking device. For this purpose, the tube wall portion, which is released in the form of a tongue, forms the blocking pawl. Released in the form of a tongue means that the tube wall is severed in the radial direction by means of an incision which in the widest sense is U-shaped. The tube wall portion released by means of the incision has to this extent in the widest sense the form of a tongue. At one end, in the region of the “tongue root”, the tube wall portion is integrally with the remaining tube wall. At the other end, in the region of the “tongue tip”, the tube wall portion has the blocking edge. The released tube wall portion is spring-elastically movable between the release position and the blocking position.

In one embodiment, the at least one blocking element is a first blocking roller which is mounted rotatably about an axis of rotation and the outer circumference of which is designed for rolling on the functional section, wherein the first blocking roller has a freewheeling direction of rotation corresponding to the advancing movement and a blocking direction which is directed in the opposite direction about the axis of rotation and corresponds to the withdrawal movement. The outer circumference of the first blocking roller interacts radially and/or tangentially with the circumferential surface of the functional section. The axis of rotation of the first blocking roller is orthogonal to the longitudinal axis of the lumen—and therefore of the functional section. The configuration of the at least one blocking element as a blocking roller, in comparison to a configuration as a blocking pawl, permits in particular a smoother advancing movement and reduced stressing of the functional section. This is because the outer circumference rolls on the functional section and/or the circumferential surface thereof during the advancing movement. Only rolling friction occurs here. By contrast, sliding friction occurs in the case of the blocking pawl. The action of friction which is reduced in this way firstly permits said smooth advancing movement and secondly reduced stressing of the functional section. No rotational movement of the blocking roller is possible in the blocking direction. Accordingly, the outer circumference then acts on the outer circumference of the functional section with a force fit and/or form fit, thereby blocking the proximal movability of the latter.

In one embodiment, the first blocking roller is guided linearly along a first guide axis on a first linear guide, wherein the first guide axis is inclined relative to a longitudinal axis of the lumen, and wherein the first blocking roller is movable along the first guide axis under the action of the functional section, as a result of which the outer circumference is movable radially outward and/or inward relative to the functional section. The first guide axis is inclined radially inward in the proximal direction relative to the longitudinal axis of the lumen. When the functional section is pulled out, the first blocking roller is moved proximally along the first guide axis, as a result of which the outer circumference of the blocking roller is positioned radially inward against the functional section. This positioning or pressing against causes blocking of the withdrawal movement.

In one embodiment, a second blocking roller is provided and positioned opposite to the first blocking roller in the radial direction of the lumen, wherein the second blocking roller is guided linearly along a second guide axis on a second linear guide, wherein the second guide axis is oriented mirror-symmetrically with respect to the longitudinal axis of the lumen in relation to the first guide axis. This embodiment of the invention permits particularly reliable blocking of the withdrawal movement. This is because the functional section, as it is being pulled out, is clamped radially between the first blocking roller and the second blocking roller. The first blocking roller and the second blocking roller preferably form a pair of blocking rollers. In a further embodiment, there are more than two blocking rollers, and they are arranged preferably in a star-shaped manner with respect to the functional section, preferably so as to form a blocking roller package.

In one embodiment, at least the first blocking roller is accommodated rotatably and/or linearly movably in the device housing. Also conceivable in principle is an integration of the first blocking roller and/or of the second blocking roller in the cannula tube or a possible cannula attachment. However, the accommodating in the device housing affords design advantages since comparatively more construction space can be provided and conventional dimensions of the cannula tube and/or of the cannula attachment can be retained.

In one embodiment, the cannula assembly comprises the medical functional section. The medical functional section is preferably a medical wire, preferably a Seldinger wire.

### BRIEF DESCRIPTION OF THE DRAWINGS

Further advantages and features of the invention follow from the claims and the description below of preferred exemplary embodiments of the invention that are illustrated with reference to the drawings.

FIG. 1 shows, in a partially cutaway, schematic perspective view, a cannula assembly, which is known from the prior art, in the region of a sharpened cannula tip together with a medical functional section in the form of a guide wire;

FIG. 2 shows, in a perspective and partially cut-open view, an embodiment of a cannula assembly according to the invention with a cannula tube, a cannula attachment and a mechanical blocking device integrated in the cannula attachment;

FIG. 3 shows an enlarged view of a detail of the cannula assembly according to FIG. 2 looking at the blocking device in a release position and during a distally directed advancing movement of a guidewire;

FIG. 4 shows the cannula assembly according to FIGS. 2 and 3 in a view corresponding to FIG. 3 in a blocking position of the blocking device and during a proximally directed withdrawal movement of the guide wire;

FIG. 5 shows, in a partially cut-open, schematic perspective view, a further embodiment of a cannula assembly according to the invention with a blocking device integrated in the cannula tube;

FIG. 6 shows an enlarged perspective view of a detail of the cannula assembly according to FIG. 5 in the region of the blocking device;

FIG. 7 shows a further perspective and partially cut-open view of a detail of the cannula assembly according to FIGS. 5 and 6 in the region of the blocking device;

FIG. 8 shows, in a partially cut-open, schematic perspective view, a further embodiment of a cannula assembly according to the invention, the blocking device of which comprises two blocking rollers;

FIG. 9 shows the cannula assembly according to FIG. 8 in a further use state; and

FIG. 10 shows the cannula assembly according to FIGS. 8 and 9 in a blocking position of the blocking device.

### DETAILED DESCRIPTION

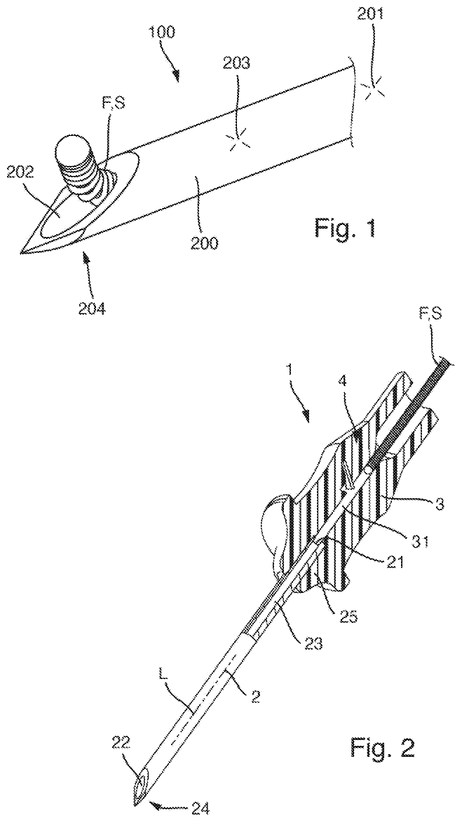

A cannula assembly 100 which is known from the prior art and has an elongate cannula tube 200 is shown in certain regions and in schematically simplified form with reference to FIG. 1. The cannula tube 200 has a proximal inlet opening 201, not illustrated specifically in the drawing, a distal outlet opening 202, a lumen 203 extending continuously between the inlet opening 201 and the outlet opening 202, and a sharpened cannula tip 204 delimiting the outlet opening 202. The sharpened cannula tip 204 facilitates puncturing of animal or human body tissue and may also be referred to as a sharpening or cannula eyelet. Furthermore, a flexible medical functional section F in the form of a guide wire S is shown with reference to FIG. 1. The guide wire S may also be referred to as a Seldinger wire and has a function and design known to those skilled in the art. The guide wire S is inserted at one end into the inlet opening 201, is advanced along the lumen 203 and protrudes on the end side out of the outlet opening 202 in the configuration shown with reference to FIG. 1. In addition, the guide wire S is shown in a state deformed by bending on the end side, in the manner in which it can be set, for example, by the guide wire S running against a tissue-side obstacle, for example a vein wall, an organ wall or the like.

Starting from the configuration shown with reference to FIG. 1, during a retraction movement of the guide wire S directed in a proximal direction, damage to the latter may occur due to the cannula tip 204. If the guide wire S is pulled out, the edge of the outlet opening 202, which edge is formed by the cannula tip 204, acts on the guide wire S in the manner of a blade. By this means, the guide wire S in the worst case can be severed, which can cause serious medical complications.

To avoid such complications, the cannula assembly 1 according to the invention which is shown with reference to FIGS. 2, 3 and 4 has a mechanical blocking device 4 which, in a manner still to be described in detail, automatically blocks a proximally directed retraction movement of the functional section F. Before the further details and the functioning of the mechanical blocking device 4 are discussed in detail, first further structural and functional features of the cannula assembly 1 according to the invention will be explained:

The cannula assembly 1 has an elongate cannula tube 2 with a proximal inlet opening 21 and a distal outlet opening 22. The proximal inlet opening 21 and the distal outlet opening 22 are connected to each other by means of a lumen 23 extending continuously. Distally, the cannula tube 2 has a sharpened cannula tip 24 which delimits the outlet opening 22. The cannula tube 2 is configured for receiving and guiding the flexible medical functional section F, which is illustrated in turn as a guide wire S with reference to FIG. 2. To this extent, the configuration of the cannula tube 2 basically corresponds to the configuration of the cannula tube 200 known from the prior art.

Furthermore, the cannula assembly 1 has a cannula attachment 3. The cannula attachment 3 supports the cannula tube 2 and is joined in a manner known to those skilled in the art to a cannula end 25 of the cannula tube 2 facing away from the cannula tip 24. The cannula attachment 3 is elongate between a proximal end, not denoted specifically, and a distal end, not denoted specifically, and has a lumen 31 which extends continuously and leads at one end into the inlet opening 21 of the cannula tube 2. The lumen 31 of the cannula attachment 3 and the lumen 23 of the cannula tube 2 are elongate coaxially with respect to each other. The remaining shaping of the cannula attachment 3 that is apparent with reference to FIG. 2 is not of primary importance regarding the present invention, and therefore further explanations are dispensed with.

As is furthermore shown with reference to FIG. 2, the guide wire S can be advanced starting from the proximal end of the cannula attachment 3 into the lumen 31 thereof and starting from the latter via the inlet opening 21 into the lumen 23 of the cannula tube 2 and from there further into a configuration going beyond the outlet opening 22. The advancing movement takes place here in the distal direction along a longitudinal axis L of the lumen 23, which, in the embodiment shown, is coaxial with respect to the longitudinal axis of the cannula tube and of the lumen 31.

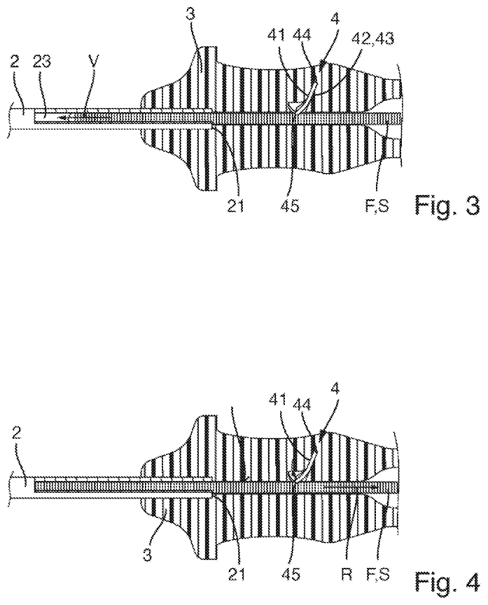

The mechanical blocking device 4 is integrated in the cannula attachment 3 in the embodiment shown with reference to FIGS. 2 to 4. In addition, the mechanical blocking device 4 has a blocking element 41 assigned to the lumen 23. The blocking element 41 is configured for mechanically interacting with the functional section F, more precisely: the guide wire S. In this case, the blocking element 41 interacts with an outer surface M of the functional section F, which may also be referred to as a circumferential surface. Depending on the movement direction of the functional section F, the blocking element 41 can be automatically transferred between a blocking position (FIG. 4) and a release position (FIG. 3). In the blocking position, the blocking element 41 blocks a proximally directed withdrawal movement of the functional section F. For this purpose, the blocking element 41 acts on the outer surface M with a force fit and/or form fit. By contrast, this action or blocking is canceled in the release position.

The arrows which can be seen with reference to FIGS. 3 and 4 symbolize a distally directed advancing movement V and a proximally directed withdrawal movement R.

In the embodiment shown, the blocking element 41 is a blocking pawl 42 which is movable spring-elastically between the blocking position and the release position. The blocking pawl 42 here is a bending spring 43 which is manufactured separately from the cannula attachment 3 and is then integrated therein. The bending spring 43 is secured at one end, at an end 44 lying radially on the outside, in a receiving cutout, not denoted specifically, of the cannula attachment 3. For example, the end 44 can be secured in the receiving cutout with a force fit, form fit and/or material bond.

In an embodiment which is not illustrated specifically in the drawing, the blocking pawl 42 and/or the bending spring 43 is formed by a portion of the cannula attachment and to this extent is integrally with the remaining portions of the cannula attachment.

Radially at the other end, the blocking pawl 42 in the form of the bending spring 43 has a blocking edge 45. The blocking edge 45 is oriented in the distal direction. During the advancing movement V, the outer surface M slides in the distal direction along the blocking edge 45. Owing to the distal alignment of the blocking edge 45, the frictional force occurring in this case brings about a force component, which is directed radially outward, on the blocking edge 45. This counteracts jamming of the blocking edge 45 on the outer surface M and ensures a smooth advancing movement V.

During a withdrawal movement R (FIG. 4), the frictional force occurring between the functional section, more precisely at the outer surface M thereof, and the blocking edge 45 causes a force component which is oriented inward in the radial direction. Said force component which is directed radially inward causes the blocking edge 45 to be pressed onto the outer surface M in the radial direction. Ultimately, a force fit and/or form fit with the outer surface M and blocking of the withdrawal movement R are thereby brought about.

Furthermore, in the illustrated embodiment, the blocking pawl 42 and/or the bending spring 43 are manufactured from a spring-elastic elastomer material. In addition, manufacturing from spring steel or the like is contemplated.

In an embodiment which is not illustrated in the drawing, the blocking pawl can alternatively or additionally be pivotally movable between the release position and the blocking position. Accordingly, a spring-elastic configuration of the blocking pawl is not necessarily required. Instead, the blocking pawl can be mounted pivotably at one end on the catheter attachment or the cannula tube by means of a pivot bearing.

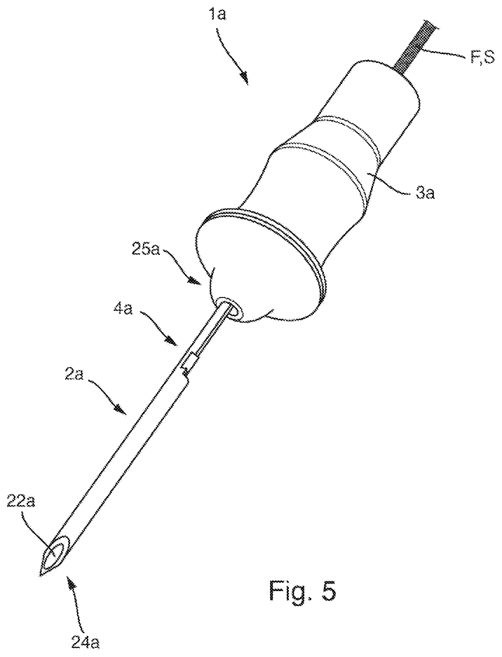

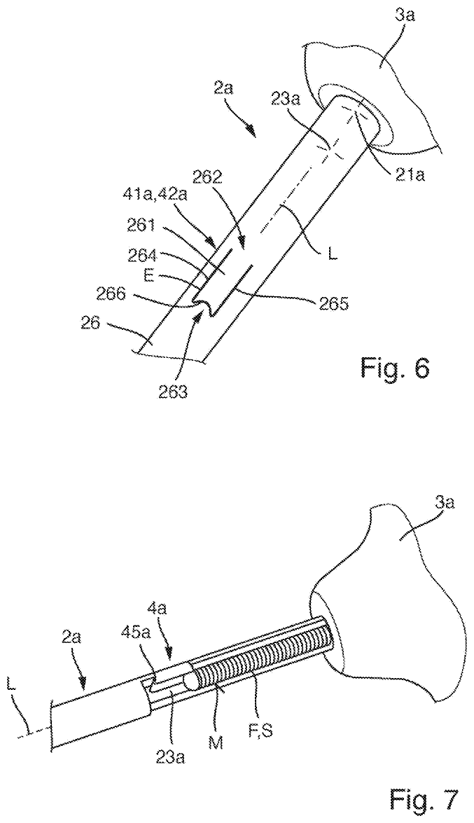

A further embodiment of a cannula assembly 1a according to the invention is shown with reference to FIGS. 5 to 7. Only essential differences of the cannula assembly 1a over the cannula assembly 1 according to FIGS. 2 to 4 will be discussed below.

An essential difference of the cannula assembly 1a is that the mechanical blocking device 4a is integrated in the cannula tube 2a. Apart from the integration of the blocking device 4a, the cannula tube 2a has a design and/or function that is substantially identical to the cannula tube 2 of the cannula assembly 1. In addition, a cannula attachment 3a is fastened in turn to the cannula end 25a of the cannula tube 2a. Except for the integrated mechanical blocking device 4 which is now not present, the cannula attachment 3a has an identical design to the cannula attachment 3. Further details regarding the cannula attachment 3a and the basic configuration of the cannula tube 2a are therefore dispensed with.

In the case of the embodiment according to FIGS. 5 to 7, the mechanical blocking device 4a also has a blocking element 41a in the form of a blocking pawl 42a. The blocking pawl 42a is formed here by a tube wall portion 261, which is released in the form of a tongue, of a tube wall 26 of the cannula tube 2a. The tube wall portion 261 is released from adjacent regions of the tube wall 26 by means of a substantially U-shaped incision extending radially through the tube wall 26. Accordingly, the tube wall portion 261 has a first end 262 connected integrally to the remaining tube wall 26, and an opposite second end 263. The second end 263 is movable in the radial direction of the cannula tube 2a relative to the remaining portions of the tube wall 26. The first end 262 is arranged proximally. The second end 263 is arranged distally. At its second end 263, the blocking pawl 42a or the tube wall portion 262 has a radially inner blocking edge 45a which is shown at least in sections with reference to FIG. 7. The blocking edge 45a interacts in a manner corresponding to the blocking edge 45 with the outer surface M of the guide wire S.

Furthermore, mutually opposite longitudinal edges 264, 265 of the tongue-shaped tube wall portion 261 extend parallel to the longitudinal axis L of the lumen 23a. In the transverse direction, and therefore in the circumferential direction of the cannula tube 2a, a transverse edge 266 extends between the two longitudinal edges 264, 265. The transverse edge 266 has a proximal indentation which is accordingly also present in the case of the blocking edge 45a. This leads to an improved form fit on the outer surface M.

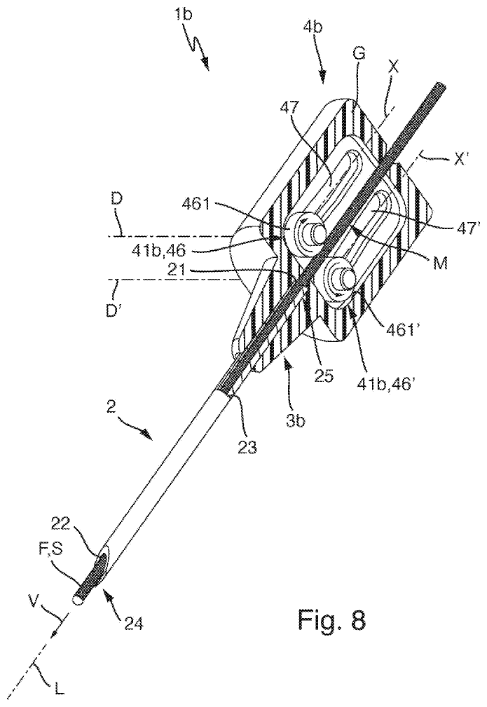

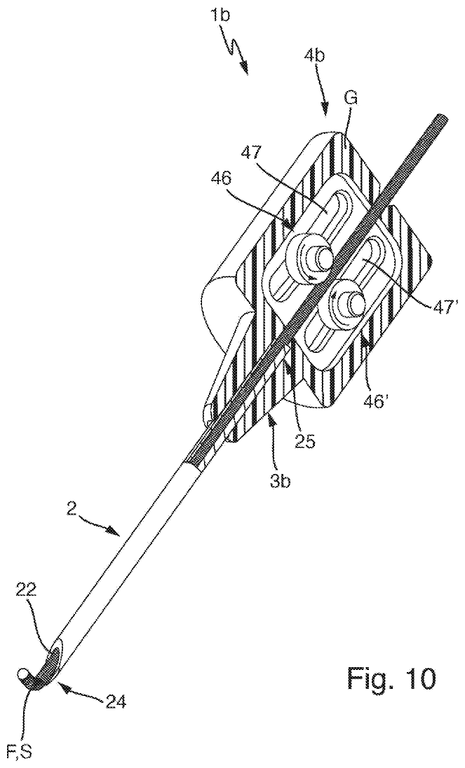

A further embodiment of a cannula assembly 1b according to the invention is shown with reference to FIGS. 8 to 10. To avoid repetitions, only essential differences of the cannula assembly 1b over the preceding embodiments will be explained.

The cannula assembly 1b has in turn a cannula tube 2 and a cannula attachment 3b. The cannula tube 2 is identical in its configuration and function to the cannula tube 2 of the embodiment according to FIGS. 2 to 4.

The cannula attachment 3b is joined in turn proximally to the cannula tube 2.

An essential difference is that the blocking device 4b of the embodiment according to FIGS. 8 to 10 is integrated neither in the cannula tube 2 nor in the cannula attachment 3b. Instead, the mechanical blocking device 4b has a device housing G in which further components and/or portions of the blocking device 4b are accommodated.

Furthermore, it will be understood that the device housing G can in principle also be assigned to the cannula attachment 3b and can be connected in particular integrally cohesively thereto. Irrespective of this, in the illustrated embodiment, the device housing G is joined releasably proximally to the cannula attachment 3b with a form fit and/or force fit in a manner not illustrated specifically in the drawing. For example, a releasable latching, plug-in or screw connection can be provided for this purpose.

A further essential difference consists in that the mechanical blocking device 4b has at least one blocking roller instead of a blocking pawl. In the embodiment which is shown, a first blocking roller 46 and a second blocking roller 46′ are present. The first blocking roller 46 and the second blocking roller 46′ form a pair of blocking rollers. The configuration and function of the two blocking rollers 46, 46′ is at least substantially identical. In addition, the two blocking rollers 46, 46′ are arranged symmetrically on both sides of the guide wire S with respect to the longitudinal axis L of the lumen 23. To avoid repetitions, the first blocking roller 46 will therefore primarily be discussed below. The disclosure in this regard also applies analogously to the second blocking roller 46′, unless stated otherwise.

The first blocking roller 46 is mounted rotatably about an axis of rotation D in the device housing G. The first blocking roller 46 has an outer circumference 461 which lies radially on the outside and is designed for rolling on the outer surface M. The first blocking roller 46 here has a freewheeling direction of rotation which corresponds to the advancing movement V and is symbolized with reference to the direction of rotation arrow, shown in FIG. 8, about the axis of rotation D.

Furthermore, the first blocking roller 46 can be moved guided linearly along a first guide axis X on a linear guide 47. The first guide axis X is inclined inward in the proximal direction relative to the longitudinal axis L. Said inclination is comparatively small and therefore cannot be readily seen with reference to the available figures. The first linear guide 47 is formed by guide tracks, not denoted specifically, which are arranged opposite one another along the axis of rotation D in the device housing G. The guide tracks interact with guide pins, not denoted specifically, of the first blocking roller 46. The guide pins are to this extent accommodated in the guide track so as to be slidable and/or rollable along the first guide axis X and are also rotatable about the axis of rotation D—which is displaceable along the first guide axis X.

The previous explanations with regard to the first blocking roller 46 apply, mutatis mutandis, also to the second blocking roller 46′. Functionally identical components and/or portions of the mechanical blocking device 4b that are assigned to the second blocking roller 46′ are identified using identical reference numbers with the addition of an apostrophe. To this extent, in particular a further or second axis of rotation D′, a second guide axis X′ and a second linear guide 47′ are present.

The further functioning of the mechanical blocking device 4b can be described as follows:

During an advancing movement V, the outer circumferences 461, 461′ roll on the outer surface M. The direction of rotation, symbolized with reference to the rotational movement arrows of FIG. 8, about the first axis of rotation D or the second axis of rotation D′ arises in each case in the process. These directions of rotation should each be understood as being freewheeling directions of rotation. The blocking rollers 46, 46′ take up a distal end position here with respect to the respective linear guide 47 or 47′. Accordingly, the guide pins, not denoted specifically, are held distally on the end side on the linear guides 47, 47′ such that no further distal movement of the blocking rollers 46, 46′ along the respective guide axis X, X′ can take place.

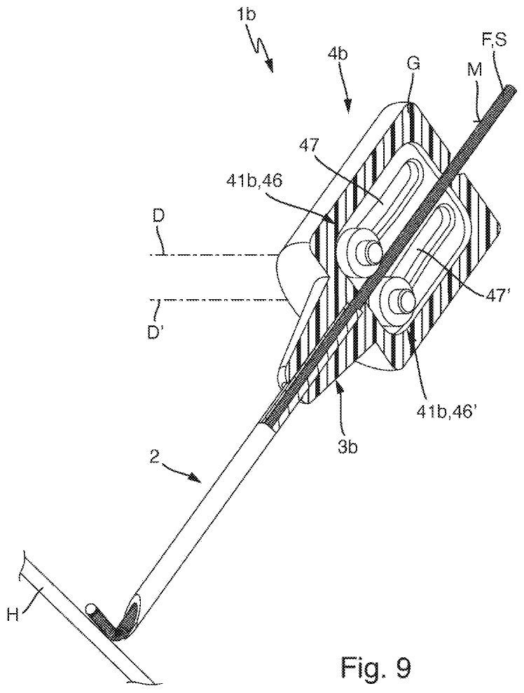

With reference to FIG. 9, a use situation of the cannula assembly 1b is shown in which the guide wire S butts against a schematically illustrated obstacle H. In this configuration, a proximal withdrawal movement of the guide wire S is conventionally required. As a result of the withdrawal movement, the blocking rollers 46, 46′ are firstly rotated in respectively opposite directions about the respective axis of rotation D, D′. At the same time, a proximally directed movement along the respective guide axis X, X′ takes place. Since the two guide axes X, X′ are inclined radially slightly inward in the proximal direction, the blocking rollers 46, 46′ are simultaneously positioned radially inward against the guide wire S as a result of their linear movement. This causes increased pressing on the respective outer circumference 461, 461′, which ultimately leads to blocking of the withdrawal movement.

Furthermore, it goes without saying that the medical functional section F or the guide wire S is not necessarily part of the cannula assemblies 1, 1a, 1b.

1.-10. (canceled)

11. A cannula assembly comprising:
a cannula tube that is elongated and has a proximal inlet opening, a distal outlet opening, a lumen extending continuously between the inlet opening and the outlet opening, and a cannula tip that is sharpened and that delimits the outlet opening; and
a blocking device with at least one blocking element,
the cannula tube being configured to receive and guide a flexible medical functional section that is insertable into the lumen and movable along the lumen,
the blocking device being assigned to the lumen,
the at least one blocking element being configured to interact with the flexible medical functional section and, depending on a movement direction of the flexible medical functional section, is automatically transferred between a blocking position, in which the at least one blocking element blocks a proximally directed withdrawal movement of the flexible medical functional section by acting on the flexible medical functional section, and a release position, in which the at least one blocking element releases a distally directed advancing movement,
the blocking device being integrated in the cannula tube.

12. The cannula assembly according to claim 11, wherein the at least one blocking element is a blocking pawl which is spring-elastically movable and/or pivotally movable between the release position and the blocking position and has a blocking edge which is oriented in a distal direction and, during the distally directed advancing movement, interacts slidably with an outer surface of the flexible medical functional section and, during the proximally directed withdrawal movement, is caused by frictional force to be positioned against the outer surface and blocks the proximally directed withdrawal movement with a force fit and/or form fit.

13. The cannula assembly according to claim 12, wherein the blocking pawl is configured as a bending spring, wherein the bending spring is secured at one end relative to the lumen and at the other end has the blocking edge.

14. The cannula assembly according to claim 12, wherein the blocking pawl is formed by a tube wall portion, that is released as a tongue, of a tube wall of the cannula tube.

15. The cannula assembly according to claim 11, further comprising the flexible medical functional section.

16. The cannula assembly according to claim 15, wherein the flexible medical functional section is a medical guide wire.

17. The cannula assembly according to claim 16, wherein the medical guide wire is a Seldinger wire.
